(19) United States
(12) Patent Application Publication
(10) Pub. No.: US 2014/0271830 A1
(43) Pub. Date: Sep. 18, 2014

(54) COMPOSITION AND METHOD FOR GRAY HAIR TREATMENT

(71) Applicant: Jacob CANTOR, Boca Raton, FL (US)

(72) Inventor: Jacob CANTOR, Boca Raton, FL (US)

(21) Appl. No.: 13/800,286

(22) Filed: Mar. 13, 2013

(51) Int. Cl. A61K 31/715; A61K 31/19

(52) U.S. Cl. CPC A61K 31/715; A61K 31/19
USPC 424/451; 514/54

(57) ABSTRACT

A treatment for gray hair is described. The treatment can include a composition for treating gray hair with an active mixture including black trap molasses and vinegar. A standard ratio of vinegar to blackstrap molasses can be at least 1:10. The treatment can also include providing an ingestible hair treatment product including the active mixture and administering a quantity of the hair treatment product to deliver an effective amount of the active mixture.

### FIELD OF THE INVENTION

This invention is directed generally to compositions and methods for hair care and, more particularly, to the treatment of gray hair.

### BACKGROUND

Gray or white hair is the result of a reduction or lack of pigmentation produced by hair follicles. Because reduced pigmentation can occur over time, gray hair is often associated with aging. Some statistics report at least 50 percent of persons over age 20 have gray hair. However, premature graying affects a number of people at much younger ages, including as young as their teens and 20s.

It can be aesthetically preferred to avoid or reduce the amount of gray hair to achieve a more youthful appearance, and gray hair treatments are in great demand. Various treatments have been used to prevent, reduce, and cover gray hair. Conventional treatments for gray hair, such as hair dye and hair makeup, are often superficial and temporary. While conventional treatments are useful in some instances, there are still numerous deficiencies and potential opportunities for new and improved, more effective and lasting treatments for gray hair.

### SUMMARY OF THE INVENTION

One of the broader forms of the present disclosure relates to a composition for treating gray hair. The composition includes an active mixture including blackstrap molasses and vinegar. A standard ratio of vinegar to blackstrap molasses can be at least 1:10.

The blackstrap molasses can include unsulphured black strap molasses. The vinegar can include apple cider vinegar. The composition can also include one or more flavoring agents. The composition can be ingestible. The composition can also include an ingestible carrier or coating containing the active mixture.

Another of the broader forms of the present disclosure relates to a method for treating gray hair. The method can include providing an ingestible hair treatment product including an active mixture and administering an effective amount of the hair treatment product. The active mixture can include blackstrap molasses and vinegar. Administering the hair treatment can include ingesting the hair treatment product on at least 30 consecutive days, or at least 60 consecutive days, or more. The amount of product can include at least 2 tablespoons of blackstrap molasses for each ingestion occasion. The amount can also include at east two tablespoons of vinegar for each ingestion occasion.

Another of the broader forms of the present disclosure relates to a kit for treating gray hair. The kit can include a composition including blackstrap molasses and vinegar and instructions for treating gray hair with the composition. The kit can also include a plurality of doses of the composition and a supply of at least 30 days, or at least 60 days, or more.

These and other embodiments are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a flow chart showing a method of making a composition for treating gray hair disclosed herein.

FIG. 2 is a flow chart showing a method of treating gray hair disclosed herein.

FIG. 3 is a schematic view of a kit for treating gray hair disclosed herein.

### DETAILED DESCRIPTION OF THE INVENTION

A treatment for gray hair is described. The treatment is unique in that it reduces or eliminates gray hair over a period of time, including promoting the return of natural hair color. As used herein, “natural hair color” refers to hair color through the natural pigmentation of hair follicles and can apply to pigmentation resulting in blonde, brown or black hair. Although the treatment is discussed herein in connection with the hair on a person's head, it is understood that the treatment can also apply to other hair types, including facial and other body hair, and other mammals besides humans.

The terminology used herein is for the purpose of describing particular embodiments only and is not intended to be limiting of the treatment. As used herein, the singular forms “a” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

The treatment can include a composition for treating gray hair. The composition can have an active mixture including blackstrap molasses and vinegar. As used herein, “blackstrap molasses” has its standard meaning in the art and includes the substance remaining after the third extraction of sugar from sugar cane. Black strap molasses can be in the form of a syrup or be further processed into other forms, such as a powder.

In some embodiments, the blackstrap molasses can be unsulphured blackstrap molasses. As used herein, “unsulphured blackstrap molasses” has its standard meaning in the art and includes blackstrap molasses produced by processing sugar cane (e.g., ripe sugar cane) where the processing does not include the addition of sulphur, which is generally added as a preservative in sulphured blackstrap molasses. In some embodiments, the blackstrap molasses can include a blackstrap molasses extract or unsulphured blackstrap molasses extract. As used herein, “extract” has its standard meaning in the art and includes a concentrated substance obtained by extracting a part of a raw material. Extracts can include liquid forms, such as tinctures and concentrates, and solid forms, such as powders.

As used herein, “vinegar” has its standard meaning in the art and includes substances produced by fermentation of dilute alcoholic liquids, including liquids obtained from fruits and other sources. The vinegar can be in the form of a liquid and can be further processed into other forms such as a tincture, or a concentrate. In some embodiments, the vinegar can be apple cider vinegar or unfiltered apple cider vinegar. In some embodiments, the vinegar can include white vinegar, sage vinegar, rosemary vinegar, apple cider vinegar, and combinations thereof. In some embodiments, the vinegar can include a vinegar extract, such as an apple cider vinegar extract or other vinegar extracts. The vinegar can be standardized to an appropriate level of acetic acid (e.g., 2-40%, 4-36%, 4-10%, 10-25%, 25%-40%, or 30%-38%).

The standard ratio of vinegar to blackstrap molasses in the active mixture is such that a biological action of the composition is increased synergistically for the promotion of natural hair color and/or treatment of gray hair. In some embodiments, the standard ratio of vinegar to blackstrap molasses in the active mixture can be at least 1:10. In other embodiments, the standard ratio can also be about 11. The standard ratio of vinegar to blackstrap molasses can also be about 1:10 or more, 1:8 or more, 1:5 or more, 1:3 or more, 1:2 or more, 1:1 or more, 2:1 or more, 4:1 or more, and about 1:9 or less, 1:5 or less, 1:2 Of less, 1:1 or less, 3:1 or less, 5:1 or less, 10:1 or less, 12:1 or less, 20:1 or less, or any combination thereof (e.g. range from 1:1 to 1:10). For example, the standard ratio of vinegar to blackstrap molasses can range from about 1:10 to 10:1, or from about 1:10 to 5:1, or from about 1:8 to 3:1, or from about 1:5 to or from about 1.3 to 3.1, or from about 1:2 to 2:1, or from about 1:1 to 6:1.

As used herein, “standard ratio” refers to the volume ratio of vinegar adjusted to 5% acidity and 79.5 Brix blackstrap molasses. When the acidity of the vinegar is adjusted or the solids content of the blackstrap molasses is adjusted, the ratio should be adjusted so that the mass ratio of acetic acid to blackstrap molasses solids remains the same. For example, if the standard ratio of vinegar to blackstrap molasses is 1:1, but the vinegar is adjusted to 20% acidity, a standard ratio of vinegar to blackstrap molasses can be produced using a 1:4 volume ratio of vinegar to blackstrap molasses.

The composition can also include additives, such as water to dilute the active mixture and flavoring agents. Flavoring agents can enhance, mask, or otherwise change the flavor of the composition. Flavoring agents can include natural and artificial flavors. For example, flavoring agents can include, without limitation, honey, sugar, artificial sweeteners, fruit, including fruit juices, vegetables, including vegetable juices, coffee, cocoa, cinnamon, ginger, similar ingredients, and combinations thereof.

The composition can be in the form of a syrup, a drink, a capsule, a lozenge, or similar forms. The composition can also include any other form suitable for oral ingestion.

In some embodiments, the composition can be a liquid, e.g. a liquid mixture. For example, the composition can include a solution, a suspension, or an emulsion. In some embodiments, the active mixture can be diluted with water or other suitable diluents. In other embodiments, the active mixture and one or more flavoring agents can be in solution. In other embodiments, the active mixture can be suspended or emulsified within one or more flavoring agents.

In some embodiments, the composition can be delivered by a capsule, a particle, or another delivery system. The composition can include a carrier or coating containing the active mixture. In one arrangement, one or both of the vinegar and blackstrap molasses can be an extract. The extracts can be encapsulated in or otherwise contained by a soluble capsule or a soluble carrier matrix. For example, the composition can include a glassy matrix formed by a blend of high and low carbohydrate polymers. As used herein, the term “high” with respect to carbohydrate polymers refers to a molecular weight greater than 1,000, and the term “low” with respect to carbohydrate polymers refers to a molecular weight of about 90-950. The composition can include about 20-80% of the high carbohydrate polymers and 20-80% of the low carbohydrate polymers, based on total weight of the carbohydrate polymers.

In other embodiments, the composition can be contained within soluble capsules formed from gelatin, hypromellose, or other suitable water soluble materials. The carrier or coating can facilitate delivery of the active mixture. For example, the carrier or coating can provide flavoring, can make the composition easier to swallow, can simplify dosing, can enhance an appearance of the composition, can extend a shelf life of the composition, and/or can provide a controlled release of the active mixture.

As shown in FIG. 1, a method 100 of making the composition for treating dray hair is also described. The method 100 can include providing blackstrap molasses 110, providing vinegar 120, and mixing the vinegar and the blackstrap molasses 130. The vinegar and blackstrap molasses can be mixed in a standard ratio, as described above, such that a biological action of the composition is increased synergistically for the promotion of natural hair color and/or treatment of gray hair. The method 100 can also include providing blackstrap molasses (e.g., unsulphured blackstrap molasses) and providing apple cider vinegar (e.g., unfiltered apple cider vinegar).

The mixing step 130 can also include providing water or other diluents, adding the vinegar and the blackstrap molasses to the water or other diluents, and mixing. The method 100 can also include mixing at least one flavoring agent with the blackstrap molasses and the vinegar 140. Mixing can include any suitable process to mix the ingredients, such as stirring, other agitation, and heating.

The method 100 can also include providing an active mixture including blackstrap molasses and vinegar and further processing of the composition 150. Further processing 150 can include preparing the active mixture for an oral delivery 150. For example, the method 100 can include encapsulating at least a portion of the active mixture into, or otherwise surrounding the active mixture with, a water soluble, ingestible carrier or coating. Further processing 150 can also include packaging the composition. The packaging step can include packaging the composition in a kit with instructions for using the composition for the treatment of gray hair. The packaging step can also include dividing the composition and/or packaging an amount of the composition into a plurality of doses. As used herein, the terms “dose” and “doses” refers to a predetermined amount of the composition, including an amount intended for user to self-administer or administer to another.

As shown in FIG. 2, a method 200 for treating gray hair using the composition is also described. The method 200 can include providing an ingestible hair treatment product 210 including the active mixture described above, and administering an effective amount of the active mixture to a person in need thereof 220. The administering step 220 can include ingesting a quantity of the hair treatment product in order to deliver an effective amount of the active mixture.

The administering step 220 can include administration of the hair treatment product over a period of time. For example, the period of time can include at least 30 days, at least 45 days, at least 60 days, at least 90 days, at least 6 months, at least 1 year, or longer.

The administering step can include administration of the hair treatment product at intervals (e.g., regular intervals). For example, the hair treatment product can be administered at intervals including, without limitation, at least twice daily, at least once daily, on alternating days, weekly, or monthly. As used herein, terms denoting administration intervals, including “daily,” refer to an intended interval and include slight deviations, for example if a user misses a day, a few days, etc.

In some embodiments, the hair treatment product can be ingested or at least 30 consecutive days. In some embodiments, the hair treatment product can be ingested daily for at least 60 consecutive days. In some embodiments, the hair treatment product can be ingested daily for at least 90 days. In some embodiments, the hair treatment product can be ingested daily for at least 6 months.

The administering step 220 can also include ingesting a predetermined quantity of the hair treatment product at each ingestion occasion. As used herein, an “ingestion occasion” refers to an individual occasion when a user ingests the composition. For example, if the product is administered once daily for one week, there will be about seven ingestion occasions or about one ingestion occasion per day for about seven days. The predetermined quantity for each ingestion occasion can be considered a single dose of the composition. In some embodiments, the quantity of the hair treatment product can include at least 2 tablespoons of blackstrap molasses in liquid form, or an equivalent measure for powder form, for each ingestion occasion. In some embodiments, the quantity of the hair treatment product can include at least 2 tablespoons of vinegar, or an equivalent measure for an extract form, for each ingestion occasion. In some embodiments, the hair treatment product can include a mixture of blackstrap molasses and vinegar within a capsule, and the quantity of hair treatment product for an ingestion occasion can be at least one capsule (e.g., one capsule, two capsules, three capsules, etc.).

In some embodiments, the method 200 can also include applying a neutralizing agent 230 after ingesting the hair treatment product. The neutralizing agent can alleviate at least some potential undesired effects of the vinegar. For example, the neutralizing agent can include baking soda, milk, and similar agents. The neutralizing agent can be ingestible. For example, a user can drink the neutralizing agent after ingesting the hair treatment product or can make a more limited application of the neutralizing agent, such as in the form of a mouth rinse.

As shown in FIG. 3, a kit 30 for treating gray hair is also described herein. The kit 30 can include the composition 10 as described above and instructions 20 for treating gray hair with the composition 10. The instructions 20 can include instructions for any of the methods or dosage regimes discussed herein for administration of the composition 10. For example, the instructions can instruct a user to ingest the composition. The instructions can also instruct a user to ingest the composition daily. The instructions can also instruct a user to ingest the composition twice daily. The instructions can also instruct a user to ingest the composition once or twice daily for at least 30 days. The instructions can also instruct a user to ingest the composition at regular intervals (e.g., morning and evening, or with each meal) until satisfactory results are achieved.

The kit 30 can also include a dose or a plurality of doses of the composition 10. The kit 30 can also include a supply of doses of the composition sufficient to cover a predetermined period of time. In some embodiments, the supply can cover at least 30 days. In some embodiments, the supply can cover at least 60 days. In some embodiments, the supply can cover at least 90 days.

The supply of the composition 10 can correspond to the instructions 20 in the kit 30. For example, the instructions 20 can instruct a user to ingest one dose of the composition 10 daily for at least 30 days and the supply can include 30 doses of the composition 10, in other examples, the instructions 20 can instruct a user to ingest one dose of the composition 10 daily for multiple months, e.g. 6 months, and the supply can include doses for the first month, e.g., 30 or 31 doses.

Beneficial effects of the composition and methods described herein include significantly improved action to reduces or eliminates gray hair and promote the return of a natural hair color. The active ingredients blackstrap molasses and vinegar, for example apple cider vinegar, also include numerous minerals and vitamins that are beneficial for a person's health. For example, blackstrap molasses includes notable amounts of potassium, copper and calcium, in addition to manganese, iron, magnesium, vitamin B6, and selenium. Other beneficial effects of the composition may include reducing symptoms associated with fatigue, sleep issues, carpal tunnel, skin problems, including acne, eczema and psoriasis, nail deficiencies, stomach issues, menstrual issues, appetite problems, sleep disorders, restless leg syndrome, low bone density and osteoporosis, joint pain, edema, nose bleeds, fibroids, high blood pressure, diabetes, arthritis, yeast infections, bladder infections, chest congestion, asthma, sore throat, cystitis, sinus infections, high cholesterol, immune system deficiencies, detoxification, migraines, hair thinning and loss, cystic fibrosis, allergies, acid reflux, warts, flatulence, the flu and colds.

### EXAMPLES

in one instance, a 20-year old male subject had between about ⅛ to 1/12 gray hair on his head. The subject's original hair color was brown. The subject ingested the composition with an active mixture of apple cider vinegar and unsulphured blackstrap molasses in a standard ratio of 1:1. In particular, each dose of the composition included 2 tbsp of apple cider vinegar, 2 tbsp of unsulphured blackstrap molasses, and 4 tbsp of water. On some occasions, a dose of the composition included 1 tbsp of honey. The subject ingested one dose of the composition daily for one year. After one year, the subject experienced a reversal of more than half of the gray hair to a mix of black and brown colored hair. Additionally, at least some of the black hair was restored to the original brown color.

In another instance, a 20-year old male subject had between about ⅛ to 1/12 gray hair on his head. The subject ingested 1 tsp of unsulphured blackstrap molasses daily for a period of time. After 3 months, the subject experienced no noticeable reduction in the amount of gray hair.

The foregoing is provided for purposes of illustrating, explaining, and describing embodiments of this invention. Modifications and adaptations to these embodiments will be apparent to those skilled in the art and may be made without departing from the scope or spirit of this invention.

I claim:

1. A composition for treating gray hair, comprising an active mixture comprising blackstrap molasses and vinegar, wherein a standard ratio of vinegar to blackstrap molasses is at least 1:10.

2. The composition according to claim 1, wherein said blackstrap molasses comprises unsulphured blackstrap molasses.

3. The composition according to claim 1, wherein said vinegar comprises apple cider vinegar.

4. The composition according to claim 1, wherein said standard ratio vinegar to blackstrap molasses ranges from about 1:10 to 5:1.

5. The composition according to claim 1, wherein said standard ratio of vinegar to blackstrap molasses ranges from about 1:3 to 3:1.

6. The composition according to claim 1, wherein said blackstrap molasses comprises an unsulphured blackstrap molasses extract.

7. The composition according to claim 1, wherein said vinegar comprises an apple cider vinegar extract.

8. The composition according to claim wherein said composition is a liquid.

9. The composition according to claim 1, wherein said composition is ingestible.

10. The composition according to claim 1, further comprising one or more flavoring agents.

11. The composition according to claim further comprising an ingestible carrier or coating containing said active mixture.

12. A kit for treating gray hair, comprising:
a composition comprising blackstrap molasses and vinegar; and
instructions for treating gray hair with said composition.

13. The kit according to claim 12, wherein said instructions comprise instructions to ingest said composition daily.

14. The kit according to claim 12, further comprising:
a plurality of doses of said composition and
a supply of at least 30 days of said composition.

15. A method of treating gray hair comprising:
providing an ingestible hair treatment product comprising an active mixture comprising blackstrap molasses and vinegar; and
administering an effective amount of said hair treatment product.

16. The method according to claim 15, wherein said administering step further comprises ingesting said hair treatment product on at least 30 consecutive days.

17. The method according to claim 15, wherein said administering step further comprises ingesting said hair treatment product daily for at least 60 consecutive days.

18. The method according to claim 15, wherein said quantity comprises at least 2 tablespoons of blackstrap molasses for each ingestion occasion.

19. The method according to claim 15, wherein said quantity comprises at least 2 tablespoons of vinegar for each ingestion occasion.

20. The method according to claim 15, further comprising applying a neutralizing agent after administering the hair treatment product.
